(19) United States
(12) Patent Application Publication
(10) Pub. No.: US 2014/0316156 A1
(43) Pub. Date: Oct. 23, 2014

(54) METHOD OF PRODUCING THERAPEUTIC AGENT

(71) Applicant: DEBORAH WARNER, HOUSTON, TX (US)

(72) Inventor: DEBORAH WARNER, HOUSTON, TX (US)

(21) Appl. No.: 14/249,837

(22) Filed: Apr. 10, 2014

Related U.S. Application Data
(60) Provisional application No. 61/813,403, filed on Apr. 18, 2013.

(51) Int. Cl. C07C 67/10

(52) U.S. Cl. CPC C07C 67/10
USPC 560/176

(57) ABSTRACT

A method for producing a therapeutic agent and the agent thus produced. This therapeutic agent, referred to as D-boramine, has the following chemical formula:[(CH₃)₃NCH₂CHOHCH₂CO₂H]₂[O₂CCHOHCHOHCO₂] and has the following empirical formula: C₁₈H₃₆N₂O₁₂. The D-boramine is produced by allowing a reaction between approximately 500 mg of L-carnitine and approximately 2.5 mg of pyridoxal 5 phosphate in the presence of approximately 10 mg of fumaric acid. The reaction creates the D-boramine molecule as the reaction product. The D-boramine molecule has an orthorhombic crystalline structure and has been shown to be useful as a therapeutic agent in treating a variety of human cancers, ailments and diseases.

### CROSS-REFERENCE TO RELATED APPLICATIONS

The present application claims priority to U.S. Provisional Application Ser. No. 61/813,403 for Method of Producing Therapeutic Agent that was filed on Apr. 18, 2013.

### BACKGROUND OF THE INVENTION

1. Field of the Invention

The present invention relates to a method of producing a therapeutic agent that may be useful in the treatment of several types of human diseases including, but not limited to, cancer, tuberculosis, XDR tuberculosis and mycoplasma and the therapeutic agent thus produced.

2. Description of the Related Art

A variety of therapeutic agents have been employed to treat or prevent breast cancer. However, none function to on the cellular level to assist the body's cells in fighting off viruses and in repairing infected cells to convert them back to healthy cells.

It is believed that the present invention may be useful in treating cancer by assisting the mitochondria of a cell to mount a proper antibody reaction to destroy the virus that is attacking the cells. The present invention also appears to increase fatty acid transport and to help the cells repair DNA.

The present invention may be useful for treatment and prevention of breast cancer and other cancers, including tumors of the brain. It also may be useful for treating or support or removing an oncogene factor in the following ailments: Ewing's sarcoma, non-Hodgkin's lymphoma, genetic mutations, change of genetic cell signals in BRCA1, BRCA2, and MTHFR, Simian virus (multiple strains), herpes simplex virus, hepatitis A, B and C, human papilloma virus (all strains), Asperger's , pleural mesothelioma, gluten intolerance, autism, infertility, Alzheimer's, tuberculosis, XDR tuberculosis and mycoplasma.

### SUMMARY OF THE INVENTION

The present invention is a method for producing a therapeutic agent and the agent thus produced. This therapeutic agent, referred to as D-boramine, has the following chemical formula:

[(CH₃)₃NCH₂CHOHCH₂CO₂H]₂[O₂CCHOHCHOHCO₂]

and has the following empirical formula:

C₁₈H₃₆N₂O₁₂.

The D-boramine is produced by allowing a reaction between approximately 500 mg of L-carnitine and approximately 2.5 mg of pyridoxal 5 phosphate in the presence of approximately 10 mg of fumaric acid. The reaction creates the D-boramine molecule having an orthorhombic crystalline structure as shown in FIG. 1. D-boramine has been shown to be useful as a therapeutic agent in treating a variety of human cancers, ailments and diseases.

### BRIEF DESCRIPTION OF THE DRAWINGS

FIG. 1 is a diagram representing the crystal structure for D-boramine, a therapeutic agent that is the subject of the present invention.

### DETAILED DESCRIPTION OF THE PREFERRED EMBODIMENT

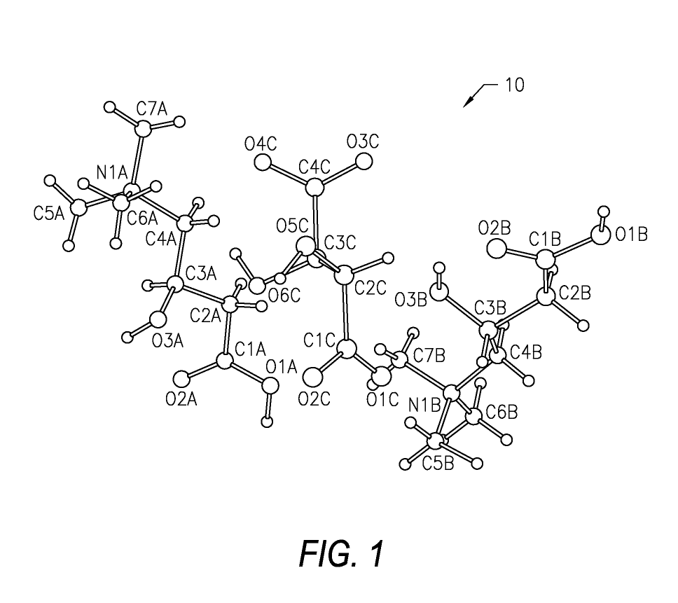

Referring now to FIG. 1, there is illustrated the structure of a molecule of D-boramine 10 which is the subject of the present invention. The present invention is a method for producing D-boramine 10 which is a therapeutic agent and the agent thus produced. D-boramine 10 has the following chemical formula:

[(CH₃)₃NCH₂CHOHCH₂CO₂H]₂[O₂CCHOHCHOHCO₂]

and has the following empirical formula:

C₁₈H₃₆N₂O₁₂.

The D-boramine 10 is produced by allowing a reaction between approximately 500 mg of L-carnitine and approximately 2.5 mg of pyridoxal 5 phosphate in the presence of approximately 10 mg of fumaric acid. The reaction proceeds immediately without the input of heat or any other outside agent.

The reaction creates the D-boramine 10 molecule having a crystalline structure as shown in FIG. 1. D-boramine 10 has been shown to be useful as a therapeutic agent in treating a variety of human cancers, ailments and diseases.

It is believed that the present invention may be useful in treating cancer by assisting the mitochondria of a cell to mount a proper antibody reaction to destroy the virus that is attacking the cells. The present invention also appears to increase fatty acid transport and to help the cells repair DNA. When the D-boramine 10 is taken by a patient, it appears to kill the viruses that are attacking the patient's cells.

The present invention may be useful for treatment and prevention of breast cancer and other cancers, including tumors of the brain. It also may be useful for treating or support or removing an oncogene factor in the following ailments: Ewing's sarcoma, non-Hodgkin's lymphoma, genetic mutations, change of genetic cell signals, Simian virus (multiple strains), herpes simplex virus, hepatitis A, B and C, human papilloma virus (all strains), Asperger's syndrome, pleural mesothelioma, gluten intolerance, autism, infertility, Alzheimer's, and BRCA, BRCAZ, HERZ cancer or cancer prevention , tuberculosis, XDR tuberculosis and mycoplasma.

D-boramine 10 may be used therapeutically in the range of 100-8,000 mg. Dosage will be dependent upon cell response testing. The D-boramine 10 may be administered orally in capsule form, or may be administered orally or intravenously in liquid form.

While the invention has been described with a certain degree of particularity, it is manifest that many changes may be made in the details of construction and the arrangement of components without departing from the spirit and scope of this disclosure. It is understood that the invention is not limited to the embodiments set forth herein for the purposes of exemplification, but is to be limited only by the scope of the attached claim or claims, including the full range of equivalency to which each element thereof is entitled.

What is claimed is:

1. A method of producing a therapeutic agent comprising:
allowing approximately 500 mg. of L-carnitine and approximately 2.5 mg. of pyridoxal 5 phosphate to react with each other in the presence of approximately 10 mg of fumaric acid to form a reaction product.

2. The method of claim 1 wherein the reaction product forms D-boramine.

3. The method of claim 1 wherein the reaction product has the following chemical formula:
[(CH₃)₃NCH₂CHOHCH₂CO₂H]₂[O₂CCHOHCHOHCO₂]

4. The method of claim 1 wherein the reaction product has the following empirical formula:
C₁₈H₃₆N₂O₁₂.

5. The method of claim 1 wherein the reaction product has a crystalline structure.

6. A therapeutic agent that is the reaction product produced by the method of claim 1.
